(19) United States
(12) Patent Application Publication
(10) Pub. No.: US 2015/0040688 A1
(43) Pub. Date: Feb. 12, 2015

(54) SYSTEMS FOR TOUCHLESS PROCESSING OF DRIED BLOOD SPOTS AND METHODS OF USING SAME

(71) Applicant: University of Washington through its Center for Commercialization, Seattle, WA (US)

(72) Inventor: Sean C. Murphy, Seattle, WA (US)

(21) Appl. No.: 14/332,225

(22) Filed: Jul. 15, 2014

Related U.S. Application Data
(60) Provisional application No. 61/864,494, filed on Aug. 9, 2013.

(51) Int. Cl. G01N 1/28; G01N 33/49

(52) U.S. Cl. CPC G01N 1/286; G01N 33/49; G01N 2001/2886
USPC 73/863

(57) ABSTRACT

The present technology relates generally to systems for processing dried blood spots (DBS) using laser cutting approaches to provide for rapid, contamination-free processing and methods of using same.

### CROSS-REFERENCE TO RELATED APPLICATION

This application claims priority to pending U.S. Provisional Application No. 61/846,494, filed Jul. 15, 2013, the entire contents of which are incorporated herein by reference and relied upon.

### TECHNICAL FIELD

The present technology relates generally to systems for touchless processing of dried blood spots, and methods of using same.

### BACKGROUND

Dried filter paper dried blood spots (“DBS”) represent attractive sample matrices for many diagnostic tests. The DBS requires only a finger, toe or heel puncture, thereby eliminating the need for venipuncture by skilled phlebotomists. If properly dried and stored, many analytes are stable across a wide range of temperatures. DBS cards generally include multiple DBS configured to store blood samples in dry form. The DBS cards are more easily transported than anti-coagulated liquid blood samples. As such, the use of DBS is increasing worldwide, especially in resource-poor regions and in clinical trial settings.

However, processing of DBS samples using conventional methods introduces risk of cross-contamination. Typically, hole punchers are used to punch a disc (hereafter called a ‘spot’) from the card into a tube. Sometimes manual manipulation with scissors and/or forceps is required. While cross-contamination is generally less problematic in chemistry-based assays (where analyte concentrations usually vary <10-fold between patients), cross-contamination can be especially problematic for molecular assays, where small amount of DNA or RNA carried over from a high positive sample can result in later false-positive results. Furthermore, where conventional automatic punching is not possible, there is significant risk of repetitive stress injuries from manual punching of DBS.

Improved systems and methods for processing DBS samples, especially when molecular assays are involved, are therefore needed.

### BRIEF DESCRIPTION OF THE DRAWINGS

Many aspects of the present technology can be better understood with reference to the following drawings. The relative dimensions in the drawings may be to scale with respect to some embodiments. With respect to other embodiments, the drawings may not be to scale. For ease of reference, throughout this disclosure identical reference numbers may be used to identify identical or at least generally similar or analogous components or features.

FIG. 1A is a perspective view of a system for touchless processing of a DBS card configured according to one embodiment of the present technology.

FIG. 1B is a perspective view of a system for touchless processing of a DBS card configured according to another embodiment of the present technology.

FIG. 1C is a perspective view of a system for touchless processing of a DBS card configured according to another embodiment of the present technology.

FIG. 1D is a perspective view of a system for touchless processing of a DBS card configured according to another embodiment of the present technology.

FIG. 1E is a perspective view of a system for touchless processing of a DBS card configured according to another embodiment of the present technology.

FIG. 1F is a perspective view of a system for touchless processing of a DBS card configured according to another embodiment of the present technology.

FIG. 1G is a perspective view of a portion of a system for touchless processing of a DBS card configured according to another embodiment of the present technology.

FIG. 2 is a perspective view of an automated system for touchless processing of multiple DBS cards configured according to one embodiment of the present technology.

FIG. 3 depicts an unused DBS card configured for use with a system for touchless processing of one or more DBS cards according to any embodiment of the present technology.

FIG. 4 depicts a calibration DBS card for use with a system for touchless processing of one or more DBS cards according to any embodiment of the present technology.

FIGS. 5A-5B show results of a validation experiment of multiplexed P. falciparum-specific qRT-PCR using standard liquid blood samples (50 microliters of whole blood). FIG. 5A illustrates linearity of the method using blood-stage parasites in whole blood across a wide range of concentrations (0.000002% parasitemia to 1% parasitemia) and detected by cycle threshold (C_{T}) analysis. The inset shows linear regression for the curves. FIG. 5B shows raw (♦) and mean (▪) values of observed nominal copy number as a function of the nominal log RNA copies per mL for 84 samples ranging from 0.000001% parasitemia to >1% parasitemia. This assay also accepts samples in DBS format (generally using a DBS size corresponding to 50 microliters of whole blood).

FIG. 6 is a plot of observed parasite concentration in blood as a function of known parasite concentration for DBS samples processed with a touchless system according to the present technology (▴), DBS samples processed by manual punch (♦), and liquid whole blood samples (o).

### DETAILED DESCRIPTION

The present technology is generally directed to systems for touchless processing of DBS and methods of using such systems. DBS processing systems configured in accordance with embodiments of the present technology are expected to reduce the risk of cross-contamination, enhance the efficacy, and/or reduce the costs associated with processing DBS.

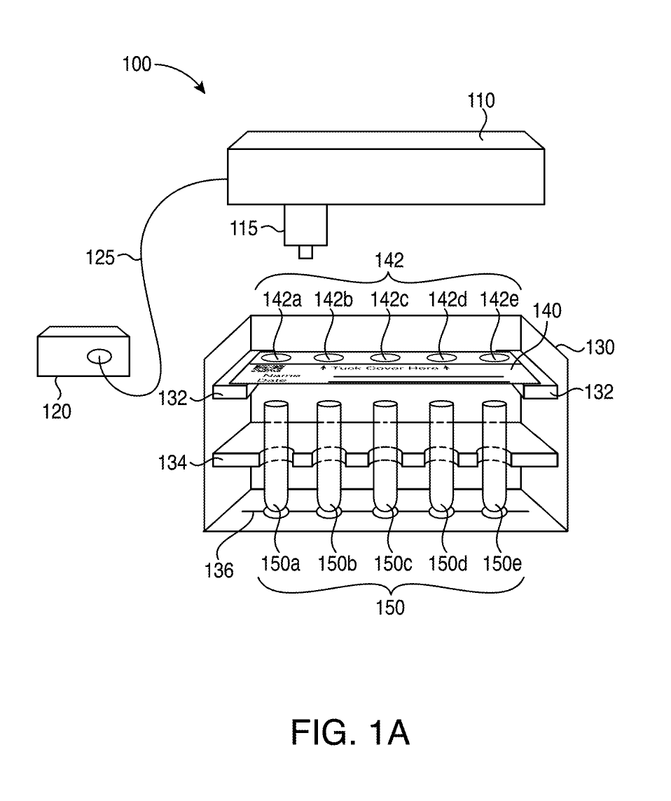

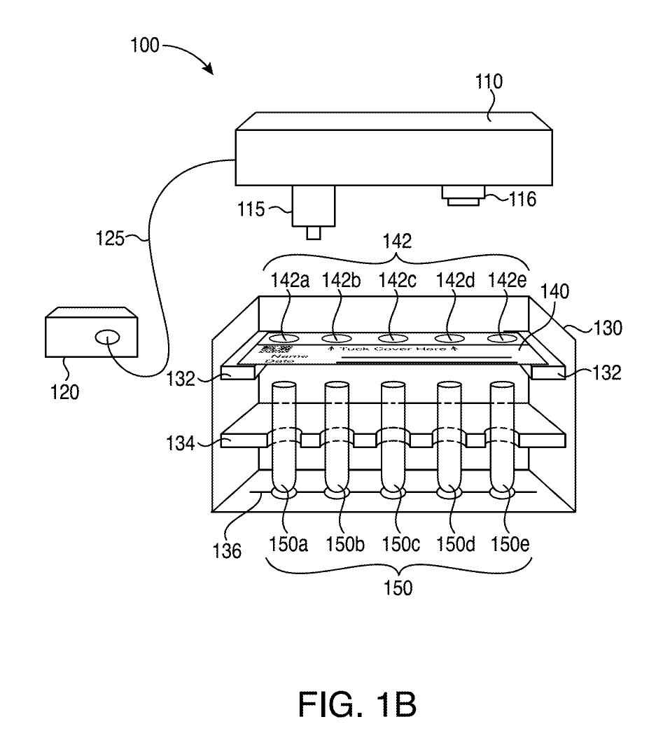

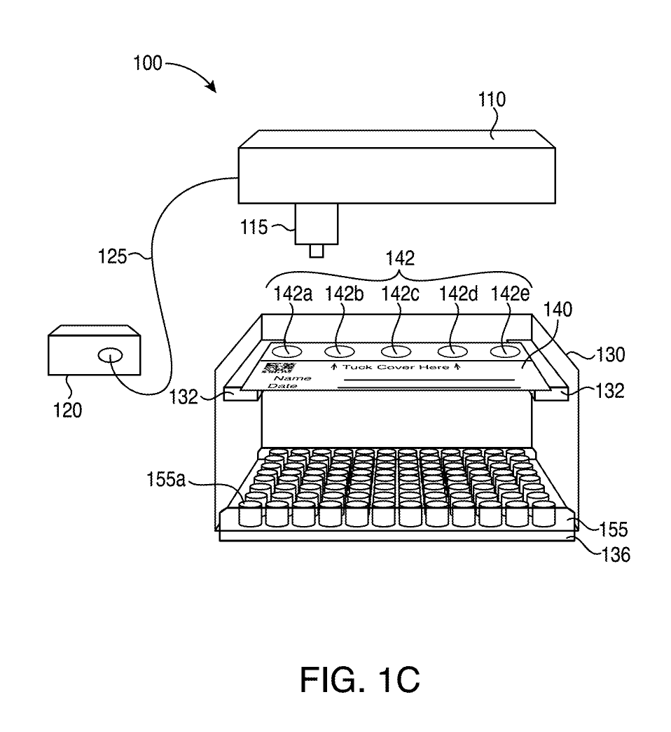

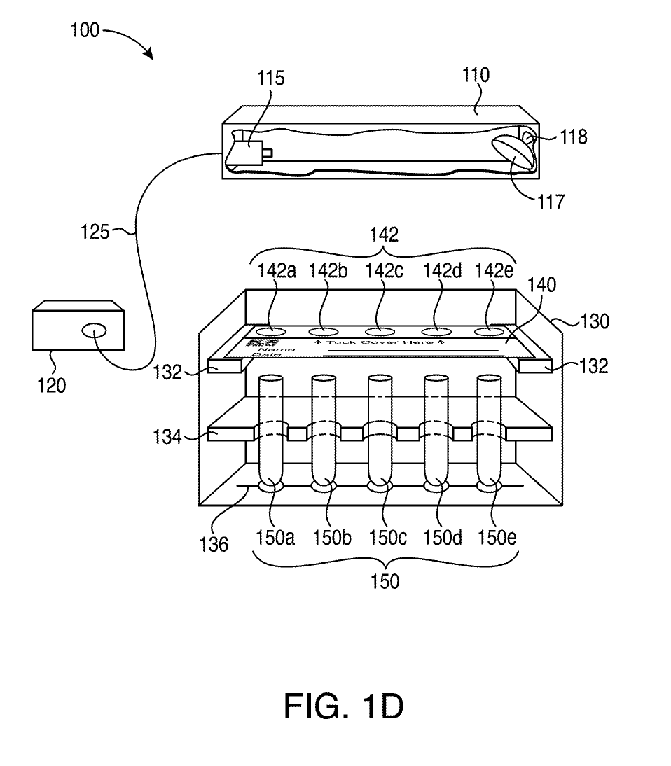

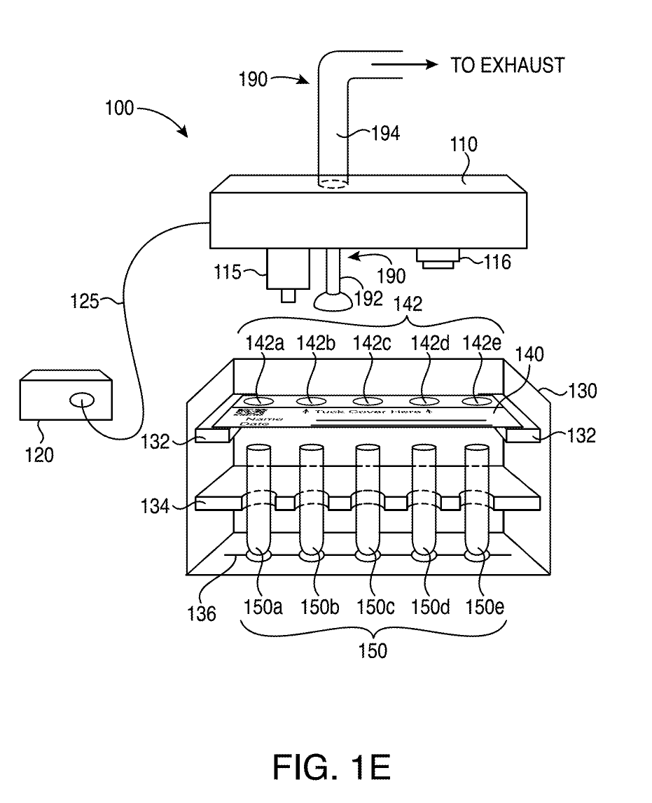

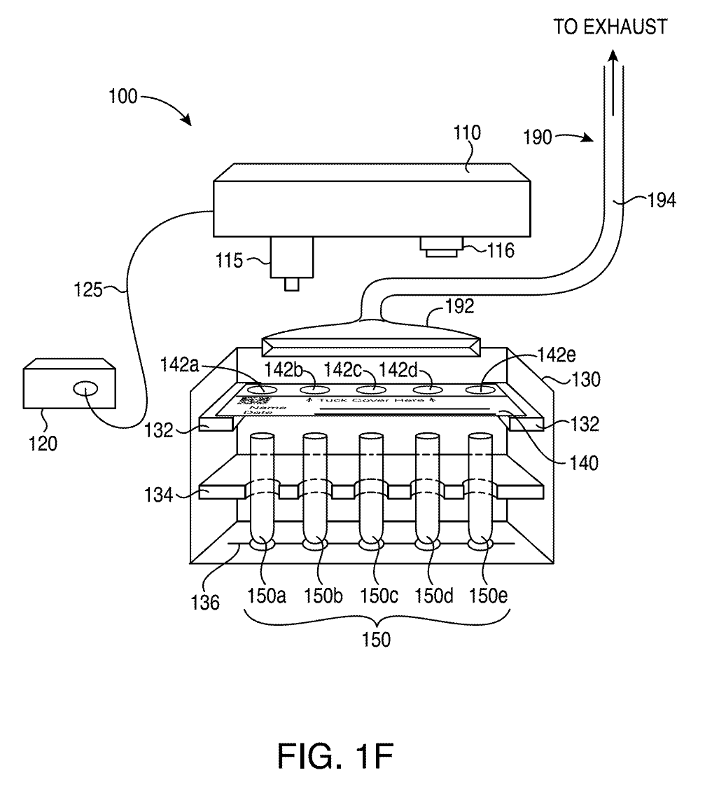

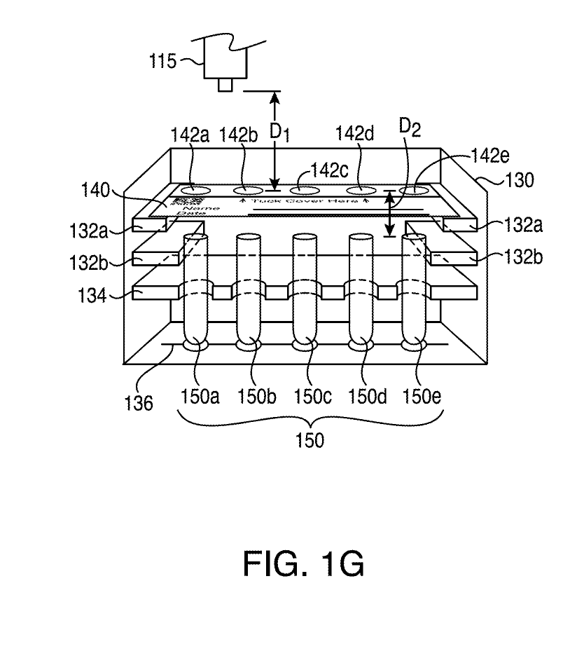

Touchless DBS processing systems consistent with the present technology may be configured to cut (e.g., laser cut) at least a portion of a DBS from a DBS card without contacting the DBS. In some embodiments, the cut portion of the DBS is deposited in a receptacle, such as a sample tube or a well of a multi-well plate, without contacting the cut portion of the DBS. Specific details of several embodiments of the present technology are described herein with reference to FIGS. 1A-7. Although many of the embodiments are described herein with respect to DBSs containing malaria or HIV material, other applications and other embodiments in addition to those described herein are within the scope of the present technology. For example, some embodiments may be useful for quantifying other infectious pathogen-derived material and/or small amounts of non-pathogen components of a blood sample without risk of contamination from processing previous DBSs. Moreover, a person of ordinary skill in the art will understand that embodiments of the present technology can have components and/or procedures in addition to those shown or described herein, and that these and other embodiments can be without several of the components and/or procedures shown or described herein without deviating from the present technology. The headings provided herein are for convenience only.

For ease of reference, throughout this disclosure identical reference numbers are used to identify similar or analogous components or features, but the use of the same reference number does not imply that the parts should be construed to be identical. Indeed, in many examples described herein, the identically-numbered parts are distinct in structure and/or function.

Generally, unless the context indicates otherwise, the terms “distal” and “proximal” within this disclosure reference a position or direction with respect to the treating clinician or clinician's surgical tool (e.g., a surgical navigation registration tool). “Distal” or “distally” are a position distant from or in a direction away from the clinician or clinician's surgical tool. “Proximal” and “proximally” are a position near or in a direction toward the clinician or clinician's surgical tool.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

Specific elements of any foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

### I. SELECTED EMBODIMENTS OF SYSTEMS FOR TOUCHLESS PROCESSING OF DBS CARDS

The present technology includes systems configured to process a DBS card by cutting at least a portion of a DBS and depositing the cutting into a receptacle, wherein the system does not physically contact the DBS (e.g., “touchless” systems), and methods of using such systems to process DBS cards with minimal or no risk of cross-contaminating samples. In some embodiments, the touchless system includes a laser configured to cut at least a portion of the DBS from the DBS card. In one embodiment, the system includes a DBS card support, a laser, a receptacle support and a controller operably connected to the laser and configured to cause the laser to cut at least a portion of a DBS from a DBS card placed in the DBS card support using a beam of light.

Referring now to FIG. 1A, some embodiments of a system 100 of the present technology (“system 100”) include a laser housing 110 including a laser 115, which is operably connected to a controller 120 and positioned proximately to a DBS card support 130. A receptacle support 134-136 positions one or more receptacles 150 in alignment with one or more DBSs 142 of a DBS card 140.

The laser 115 can be any laser capable of cutting through the DBS card 140. In general, higher powered lasers are capable of cutting the DBS card 140 while producing a minimal amount of charring and/or smoke byproduct. However, lower powered lasers are also suitable and particularly advantageous for portable embodiments of the systems described herein. In some embodiments, the laser 115 is stabilized by a laser housing 110. The laser housing 110 may include mechanisms for enabling the laser 115 to move in any direction (e.g., x, y, and/or z) relative to the DBS card support 130, and may also enable the laser 115 to rotate in one or more axes relative to the DBS card support 130. The laser 115 is operably connected to the controller 120, for example via a wired or wireless connection 125, which may control power and/or movement of the laser 115, for example according to a set of instructions stored on a memory device incorporated into the controller 120. In some embodiments, the laser housing 110 includes a cabinet, a box, a rack, a bracket, an enclosure, or any other suitable support framework for stabilizing the position of the laser 115 relative to the DBS card support 130.

The DBS card support 130 is positioned proximately to the laser 115 such that a beam emitted by the laser 115 can accurately and effectively cut a shape out of one or more of the DBSs 142 on the DBS card 140. In some embodiments, the DBS card support 130 includes one or more pairs of DBS card mounts 132 configured to enable accurate placement of DBS cards 140 in a consistent position relative to the laser 115. In the embodiment shown in FIG. 1A, the DBS card support 130 holds the DBS card 140 in a generally horizontal position under the laser 115. Other configurations are possible, however, as will be readily recognized by one of ordinary skill in the art. For example, the DBS card support 130 may in some embodiments hold the DBS card 140 in a position that is not generally horizontal, such as skewed (e.g., on an angle relative to horizontal), and the laser 115 can be positioned directly above the DBS card support 130, or alternatively can be positioned to the side or below the DBS card 140.

The receptacle support 134-136 can be in any configuration suitable for holding one or more receptacles 150 in alignment with the DBSs 142. In the embodiment shown in FIG. 1A, for example, the receptacle support 134-136 includes a base 136 and a rack 134 configured to hold a series of receptacles 142a-e generally in alignment below a series of DBSs 142a-e on the DBS card 140. Other configurations are possible and remain within the scope of the present technology, as will be recognized by a person having ordinary skill in the relevant art. For example, the receptacle support 134-136 may be configured to hold a single receptacle 150 in alignment with more than one DBS 142 of the DBS card 140. Such embodiments are particularly useful when multiple DBSs 142 are pooled into a single receptacle 150 in order to perform batch analysis or when a desired assay requires more blood sample than is present in a single DBS 142.

The controller 120 may be configured to cause the laser 115 to emit a beam that contacts the surface of the DBS card 140 at a particular location. For example, the controller 120 in some embodiments may cause the laser 115 to emit a beam that contacts the surface of the DBS card 140 at one or more of DBSs 142a-e. The controller 120 may also be configured to cause the laser 115 to emit a beam in a pattern that, upon completion, causes at least a portion of the DBS card 140 to automatically separate from the DBS card 140. In some embodiments, the pattern is a regular or common shape such as a circle, oval, ellipse, polygon, triangle, quadrilateral, square, rectangle, rhombus, parallelogram, trapezoid, pentagon, hexagon, heptagon, octagon, nonagon, decagon, etc. In some embodiments, the pattern is an irregular curved shape or an irregular polygon. In some embodiments, the pattern is a combination of curves and straight lines.

The system 100 is configured to enable the excised portions of the DBS 140a-e to be deposited into a receptacle 150a-e. In some embodiments, the excised portion of the DBS 140a-e fall into the receptacle 150a-e via gravity. In some embodiments, the excised portion of the DBS 140a-e is directed into the receptacle 150a-e by another touchless force, such as forced gas (e.g., a jet or puff of air or an inert gas such as nitrogen, argon, etc.).

The controller 120 may be configured to select a beam pattern based on one or more parameters of the DBS card 140 and/or of the assay to be performed on the DBS. For example, the controller 120 may be configured to select a beam pattern that cuts a portion of the DBS card 140 that has a predetermined area. In some embodiments the controller 120 is configured to select a beam pattern that cuts a portion of the DBS card 140 that has an area of about 10 mm² to about 200 mm², about 10 mm² to about 100 mm², about 20 mm² to about 80 mm², or about 50 mm² to about 75 mm², for example 10 mm², about 11 mm², about 12 mm², about 13 mm², about 14 mm², about 15 mm², about 16 mm², about 17 mm², about 18 mm², about 19 mm², about 20 mm², about 21 mm², about 22 mm², about 23 mm², about 24 mm², about 25 mm², about 26 mm², about 27 mm², about 28 mm², about 29 mm², about 30 mm², about 31 mm², about 32 mm², about 33 mm², about 34 mm², about 35 mm², about 36 mm², about 37 mm², about 38 mm², about 39 mm², about 40 mm², about 41 mm², about 42 mm², about 43 mm², about 44 mm², about 45 mm², about 46 mm², about 47 mm², about 48 mm², about 49 mm², about 50 mm², about 51 mm², about 52 mm², about 53 mm², about 54 mm², about 55 mm², about 56 mm², about 57 mm², about 58 mm², about 59 mm², about 60 mm², about 61 mm², about 62 mm², about 63 mm², about 64 mm², about 65 mm², about 66 mm², about 67 mm², about 68 mm², about 69 mm², about 70 mm², about 71 mm², about 72 mm², about 73 mm², about 74 mm², about 75 mm², about 76 mm², about 77 mm², about 78 mm², about 79 mm², about 80 mm², about 81 mm², about 82 mm², about 83 mm², about 84 mm², about 85 mm², about 86 mm², about 87 mm², about 88 mm², about 89 mm², about 90 mm², about 91 mm², about 92 mm², about 93 mm², about 94 mm², about 95 mm², about 96 mm², about 97 mm², about 98 mm², about 99 mm², about 100 mm², about 101 mm², about 102 mm², about 103 mm², about 104 mm², about 105 mm², about 106 mm², about 107 mm², about 108 mm², about 109 mm², about 110 mm², about 111 mm², about 112 mm², about 113 mm², about 114 mm², about 115 mm², about 116 mm², about 117 mm², about 118 mm², about 119 mm², about 120 mm², about 121 mm², about 122 mm², about 123 mm², about 124 mm², about 125 mm², about 126 mm², about 127 mm², about 128 mm², about 129 mm², about 130 mm², about 131 mm², about 132 mm², about 133 mm², about 134 mm², about 135 mm², about 136 mm², about 137 mm², about 138 mm², about 139 mm², about 140 mm², about 141 mm², about 142 mm², about 143 mm², about 144 mm², about 145 mm², about 146 mm², about 147 mm², about 148 mm², about 149 mm², about 150 mm², about 151 mm², about 152 mm², about 153 mm², about 154 mm², about 155 mm², about 156 mm², about 157 mm², about 158 mm², about 159 mm², about 160 mm², about 161 mm², about 162 mm², about 163 mm², about 164 mm², about 165 mm², about 166 mm², about 167 mm², about 168 mm², about 169 mm², about 170 mm², about 171 mm², about 172 mm², about 173 mm², about 174 mm², about 175 mm², about 176 mm², about 177 mm², about 178 mm², about 179 mm², about 180 mm², about 181 mm², about 182 mm², about 183 mm², about 184 mm², about 185 mm², about 186 mm², about 187 mm², about 188 mm², about 189 mm², about 190 mm², about 191 mm², about 192 mm², about 193 mm², about 194 mm², about 195 mm², about 196 mm², about 197 mm², about 198 mm², about 199 mm², or about 200 mm². In some embodiments, the cut area of the portion of the DBS card is divided between two or more cuttings, for example in two cuttings, three cuttings, four cuttings, five cuttings, six cuttings, seven cuttings, eight cuttings, nine cuttings, ten cuttings, or more than ten cuttings.

FIG. 1B shows another embodiment of system 100 generally as described above, and further including a camera 116. In the illustrated embodiment, the camera 116 is mounted to the laser housing 110. However, the camera 116 may be positioned in any location within system 100 suitable for obtaining an image of at least a portion of the DBS card 140. The camera 116 is operably connected to the controller 120, which in some embodiments includes instructions for obtaining and processing an image from camera 116. In some embodiments, the instructions are stored on a memory device incorporated in the controller 120. In some embodiments, the controller 120 is configured to obtain an image of the DBS card 140 and then determine a location on the DBS card 140 to be cut by the laser 115 based at least in part on the image of the DBS card 140 obtained by the camera 116. In some embodiments, the controller 120 is additionally configured to determine a shape to be cut from the DBS card 140 based at least in part on the image of the DBS card 140 obtained by the camera 116.

Referring now to FIG. 1C, the receptacle support 136 may be configured to support a multi-well plate 155, such as a 6-well plate, a 12-well plate, a 24-well plate, a 48-well plate, a 96-well plate, etc. In the embodiment illustrated in FIG. 1C, the receptacle 155 is a 96-well plate including eight rows of twelve wells 155a. In some embodiments, the receptacle support 136 is configured to move the position of the multi-well plate 155 along the x-axis, the y-axis, and/or the z-axis relative to the DBS card support 130. In some such embodiments, the receptacle support 136 is operably connected to the controller 120, which may include instructions for moving the position of the multi-well plate 155. The instructions may be stored in a memory device incorporated within the controller 120. Such embodiments enable the system 100 to cut smaller portions of a DBS 142 from the DBS card 140, for example when only a small amount of the blood sample is required to perform a desired assay. In some embodiments, the receptacle 155 is a 96-well plate, and the system 100 is configured to cause the laser 115 to cut a portion of a DBS 142 having an area of about 7 mm² (e.g., a circle having a 3 mm diameter).

Referring now to FIG. 1D, the laser housing 110 may include one or more mirrors 117 configured to adjustably reflect a beam from the laser 115 onto the DBS card 140. In such embodiments, the laser 115 may be configured to be stationary relative to the DBS support 130, whereas the mirrors 117 may each be operably connected to a motor 118. The motors 118 may be operably connected to the controller 120, which may include instructions for adjusting the position (e.g., angle) of the mirrors 117 relative to the laser 115 in order to reflect the beam onto the DBS card 140 at a location (e.g., at one or more DBSs 142) and in a pattern (e.g., a circle) to affect cutting of at least a portion of the DBS 142.

As shown in FIGS. 1E-1F, the system 100 may additionally include an exhaust 190 configured to collect and vent fumes generated by the laser cutting of the DBS card 140. The exhaust 190 includes a fume collector portion 192 and a vent portion 194 connected to the collector portion 192 and configured to safely release the collected fumes, for example in a chemical fume hood or other suitable exhaust outlet. In some embodiments (e.g., FIG. 1E), the exhaust 190 is incorporated into the laser housing 110. Such embodiments are advantageous in that the fume collection portion 192 is automatically located proximate to the portion of the DBS card 140 from which the fumes emanate. In addition, the localized nature of the proximate position enables effective fume removal by a relatively smaller amount of airflow/power draw. In other embodiments (e.g., FIG. 1F), the exhaust 190 is located adjacent to or incorporated in the DBS card support 130. For example, the fume collector portion 192 may extend substantially the entire width of the DBS card support 130 to provide substantially even and constant fume removal across the width of the DBS card 140. Such embodiments are advantageous in that the fume collector portion 192 cannot impede the path of the laser beam.

Referring now to FIG. 1G, systems configured according to the present technology are arranged such that the lowest point of the laser 115 is located at a predetermined distance D₁ from the surface of the DBS card 140. In some embodiments, the distance D₁ is about 1 cm to about 20 cm, about 2 cm to about 18 cm, about 12 cm to about 16 cm, or about 5 cm to about 10 cm, for example about 1 cm, about 2 cm, about 3 cm, about 4 cm, about 5 cm, about 6 cm, about 7 cm, about 8 cm, about 9 cm, about 10 cm, about 11 cm, about 12 cm, about 13 cm, about 14 cm, about 15 cm, about 16 cm, about 17 cm, about 18 cm, about 19 cm, or about 20 cm. Larger values of D₁ correspond to larger clearances between the DBS card 140 and the bottom of the laser 115 or the laser housing 110 which may be advantageous when a stack of DBS cards 140 is processed rapidly. Conversely, smaller values of D₁ correspond to less clearance, but provide a higher certainty that the beam from the laser 115 will contact the DBS card 140 in the desired location and in the desired pattern.

As shown in FIG. 1G, the DBS card support 130 is configured to support the DBS card 140 above the receptacles 150 at a predetermined distance D₂. One of skill in the art will readily recognize that the configuration of the DBS card support 130 may vary depending on the dimensions of the receptacles 150 to be used. In general, however, the distance D₂ is short enough to ensure that the portion of the DBS card 140 excised by the laser 115 is accurately and repeatably deposited into the desired receptacle 150 (e.g., such that a portion of one of DBSs 142a-e falls into one of receptacles 150a-e, respectively). To enable accurate and repeatable depositing of excised DBS samples into receptacles 150, the DBS card support may have any suitable shape and size. In addition, in some embodiments the DBS card support includes more than one DBS card mount 132. For example, the DBS card support 130 shown in FIG. 1G includes two sets of opposed DBS card mounts 132a and 132b, which enables the DBS card support 130 to position a DBS card 140 at a height D₂ over relatively tall receptacles 150 (e.g., using DBS card mounts 132a), and also enables the DBS card support 130 to position a DBS card 140 at a height D₂ over relatively short receptacles 150 (e.g., using DBS card mounts 132b). Other configurations for the DBS card support 130 are possible and are within the scope of the present disclosure.

Alternatively or in addition to the configurations described above, the controller 120 may be configured to store information about the DBS card 140 (e.g., identifying information about the subject and/or the blood sample), the location of the portion of the DBS 142 to be excised, the pattern of light beam to be executed by the laser 115, the power voltage and/or amperage supplied to the laser 115, the image obtained by the camera 116, notes from an operator of the system 100, and/or any error messages (e.g., power faults, laser faults, and/or possible defects in the DBS card 140a determined from the image obtained by the camera 116) that are generated by the system 100 during processing of the DBS card 140. In some embodiments, the controller 120 is configured to store the information in association with a unique identifier assigned to the DBS card 140, for example a machine-readable feature such as a 1-dimensional bar code, a 2-dimensional bar code, an RFID code, or any other suitable identifier.

### II. SELECTED EMBODIMENTS OF SYSTEMS FOR TOUCHLESS AUTOMATIC PROCESSING OF MULTIPLE DBS CARDS

The present technology also includes systems configured to process multiple DBS cards by cutting at least a portion of a DBS from each DBS card without physically contacting the DBS, and depositing each cutting into a receptacle, wherein the system does not physically contact the DBS, and methods of using such systems to process DBS cards with minimal or no risk of cross-contaminating samples. In some embodiments, the touchless system includes a laser configured to cut at least a portion of the DBS from the DBS card using a beam of light.

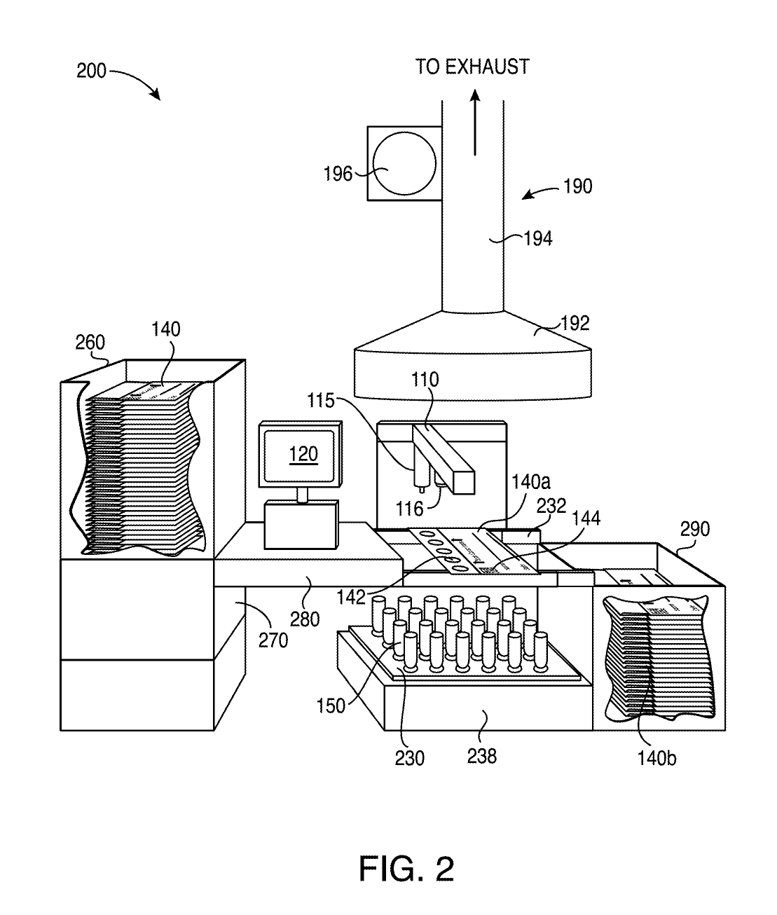

Referring now to FIG. 2, some embodiments of an automated touchless system 200 of the present technology (“system 200”) include a laser configured to cut at least a portion of the DBS from each of a plurality of DBS cards. In one embodiment, the system includes a DBS card hopper 260, a DBS card support 232, a laser 115, a DBS card feeder 280, a receptacle support 230, a DBS card depository 290, and a controller 120 operably connected to the DBS card feeder 280, the DBS card support 232 and the laser 115, and configured to cause the DBS card feeder 280 to select a DBS card 140a from among the DBS cards 140 in the DBS card hopper 260 and feed the selected DBS card 140a to the DBS card support 232, to cause a receptacle 150 to be positioned under a DBS 142 of the DBS card 140a, to cause the laser 115 to cut at least a portion of the DBS 142 from the DBS card 140a using a beam of light, and to cause the DBS card 140a to be deposited in the DBS card depository 290.

Systems 200 for automated touchless processing of DBS cards 140 may include components similar or identical to those described above with respect to touchless processing systems 100. For example, the laser 115 may be configured similarly or identically in system 200 as described above with respect to system 100. Similarly, the system 200 may include a laser housing 110 and/or a camera 116 configured similarly or identically to laser housing 110 and camera 116 as described above with respect to system 100.

The system 200 may include a DBS card hopper 260 configured to hold multiple DBS cards 140, for example, in a stack. The DBS card hopper 260 may include walls, edges, guides, rails, rollers, trays, or other components for storing the DBS cards 140 without physically contacting any DBSs 142. For example, the DBS card hopper 260 does not include any rollers, guides, treads, or other components in locations that are likely to come into contact with a DBS 142 on a DBS card 140 during the storage process. Typically, DBS cards 140 are stored in a folded configuration to prevent cross-contamination from contact with other DBS cards 140. In the folded configuration, at least one additional layer of DBS card material separate each layer of DBSs 142. The DBS cards 140 must be unfolded to expose the DBSs 142 before processing. Accordingly, in some embodiments, the DBS cards 140 are placed in the DBS card hopper 260 in an unfolded configuration. In other embodiments, the DBS cards 140 are placed in the DBS card hopper 260 in a folded configuration, for example to prevent cross-contamination caused by physical contact of a DBS 142 from one DBS card 140 with a DBS 142 from an adjacent DBS card 140. In such embodiments, the DBS card hopper 260 may include a DBS card unfolder 270 configured to accept a folded DBS card 140 from the DBS card hopper portion 260 and unfold the DBS card 140.

The DBS card feeder 280 is configured to accept a DBS card 140 (e.g., in an unfolded configuration) from the DBS card hopper 260 or the DBS card unfolder 270. The DBS card feeder 280 may include rollers, treads, gears, or other mechanisms for transporting the DBS card 140 without physically contacting any DBSs 142. For example, the DBS card feeder 280 does not include any rollers, guides, treads, or other components in locations that are likely to come into contact with a DBS 142 on a DBS card 140 during the transport process. To affect transport of the DBS card 140 from the DBS card hopper 260 and/or the DBS card unfolder 270, the DBS card feeder 280 may include opposing sets of rollers (e.g., motorized rollers) configured to pinch the edges of the DBS card 140 in the margin between the edge of the DBS card 140 and the DBSs 142 nearest the edge. In such embodiments, the DBS card feeder 280 contacts only portions of the DBS card 140 that do not include dried blood, thus reducing or eliminating the risk of cross-contaminating one DBS card 140 with dried blood from another DBS card 140.

The DBS card support 232 is configured to accept a DBS card 140 from the DBS card feeder 280 and position the DBS card 140a to be cut by the laser 115, and to enable the excised portion of the DBS 142 to be placed in a receptacle 150. Similar to the DBS card hopper 260, the DBS card unfolder 270, and the DBS card feeder 280, the DBS card support 232 may include walls, edges, guides, rails, rollers, trays, or other components for accurately and repeatably positioning the DBS card 140a without physically contacting any DBSs 142. For example, the DBS card support 232 may include two opposing rails with rollers (e.g., motorized rollers) and/or guides configured to receive the edges of the DBS card 140a and position the DBS card 140a at a predetermined location relative to the laser 115 and/or the receptacles 150. The DBS card support 232 may also be configured to transport the DBS card 140a to the DBS card depository 290 after the laser completes cutting a portion of the DBS 142. Alternatively, the DBS card depository may include a feeder portion configured to retrieve the DBS card 140a as described more fully below.

In some embodiments, the DBS card support 232 is configured to adjust the distance D₁ between the DBS card 140a and the laser 115, and/or the distance D₂ between the DBS card 140a and the receptacles 150. In other embodiments, the DBS card support 232 positions the DBS card 140a at a fixed elevation, and the laser 115/laser housing 110 can be adjusted to provide the desired distance D₁, as described more fully with respect to FIG. 1G above. Similarly, in some embodiments, the DBS card support 232 positions the DBS card 140a at a fixed elevation, and the receptacle support 230 is adjustable to provide the desired distance D₂ to the DBS 142, as described more fully below.

In embodiments wherein the DBS card support 232 is configured to (i) transport the DBS card 140a from the DBS card feeder 280, (ii) transport the DBS card 140a to the DBS card depository 290, and/or (iii) adjust the position of the DBS card 140a relative to the laser 115 and/or to the receptacle support 230, the DBS card support 232 may be operably connected to the controller 120, which may control power and/or movement of the DBS card support 232, for example according to a set of instructions stored on a memory device incorporated into the controller 120. In some embodiments, the position of the DBS card support 232 is predetermined based at least in part on information about the DBSs 142 obtained by the camera 116. In some embodiments, the position of the DBS card support 232 is predetermined based at least in part on information about the assay to be performed on the DBS, and/or the size and/or shape of the light beam pattern to be executed by the laser 115.

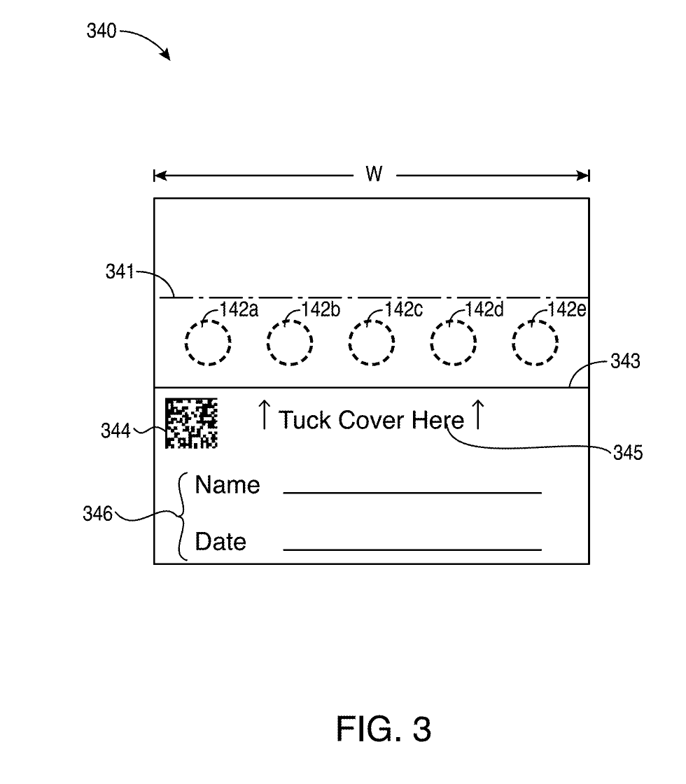

Although FIG. 2 shows the DBS card support 232 positioning the DBS card 140a in an orientation wherein the DBSs 142 are generally orthogonal to the direction the DBS card 140/140a travels. One advantage of this configuration is that the DBS card 140/140a is secured in along edges that are orthogonal to folds in the DBS card (see, e.g., fold 341 in FIG. 3). As a result, the DBS card 140a is secured along the edges that are separated by a substantially constant distance (see, e.g., width W of FIG. 3). This reduces the risk that a DBS card 140 will misfeed through any of the motorized components. In addition, this orientation provides the DBS card 140a such that the surface of the DBS card 140a is arranged in a predictable angle relative to the laser 115. This provides the additional benefits of simplifying the calculations required to determine a cutting pattern to be executed by the laser by eliminating one of the three dimensions to be factored.

In other embodiments, the DBS card hopper 260, the DBS card unfolder 270, the DBS card feeder 280, the DBS card support 232, and the DBS card depository 290 are each configured to transport and support the DBS card 140/140a in a configuration other than that shown in FIG. 2, for example in an orientation in which the DBSs 142 are substantially parallel with the direction of travel. In such embodiments, the controller 120 may be configured to (i) determine an angle of orientation of the surface of the DBS card 140a (e.g., using the camera 116 and the observed difference between the DBS shapes 142 and the actual shape, such as the circles shown in FIG. 2), and (ii) determine a location and pattern to be executed by the laser 115 in order to excise a portion of the DBS 142 having a predetermined area. In one such embodiment, the controller 120 may include instructions for adjusting a preselected pattern for cutting the DBS 142 to account for the determined angle of the surface of the DBS card 140a relative to the laser 115. For example and without limitation, the controller 120 may be configured to elongate or constrict a preselected circular light beam pattern in one or more dimensions to provide a modified light beam pattern such as an oval or ellipsis in order to accommodate an observed deflection or deviation in the angle of the surface of the DBS card 140a relative to the laser 115 and/or to the DBS card support 232.

The receptacle support 230 is configured to hold one or more receptacles 150 similar to receptacle support 130/136 as described above with respect to FIGS. 1A-1G. In some embodiments, the receptacle support 230 includes a positioner 238 which is configured to move the receptacle support 230 in the x-axis, the y-axis, or the z-axis relative to the DBS card 140a. In such embodiments, the receptacle support 230 enables positioning of a predetermined receptacle 150 in alignment with a predetermined DBS 142 and at a distance D₂ that enables the excised portion of the DBS 142 to be accurately deposited (e.g., fall) into the predetermined receptacle 150. In such embodiments, the receptacle support 230 and/or the positioner 238 is operably connected to the controller 120, which may control power and/or movement of the positioner 238, for example according to a set of instructions stored on a memory device incorporated into the controller 120. In some embodiments, the position of the positioner 238 is predetermined based at least in part on information about the DBSs 142 obtained by the camera 116. In some embodiments, the position of the positioner 238 is predetermined based at least in part on information about the assay to be performed on the DBS, and/or the size and/or shape of the light beam pattern to be executed by the laser 115.

The DBS card depository 290 is operably connected to the DBS card support 232, and is configured to store one or more DBS cards 140b after they have been processed. In some embodiments, the DBS card depository receives the DBS card 140a from the DBS card support 232. In other embodiments, the DBS card depository retrieves the DBS card 140a from the DBS card support 232 and stores the retrieved DBS card 140b. In such embodiments, the DBS card depository 290 may include rollers, gears, treads, or any other suitable transport mechanism for transporting the DBS card 140a from the DBS card support 232 to the DBS card depository 290. In such embodiments, the DBS card depository 290 is operably connected to the controller 120, which may control power and/or movement of the components of the DBS card depository 290, for example according to a set of instructions stored on a memory device incorporated into the controller 120.

In some embodiments, the system 200 additionally includes an exhaust system 190 configured to intake fumes generated by the touchless system 200 and vent the fumes to an appropriate exhaust location. In some embodiments, the exhaust system 190 includes a fume collector portion 192 and a vent portion 194 connected to the collector portion 192 and configured to safely release the collected fumes, such as described above with respect to FIGS. 1E-1F. In some embodiments, the exhaust system 190 additionally includes a blower 196 configured to draw air into the fume collector portion 192 and through the vent portion 194. The blower 196 may be operably connected to the controller 120, which may control power to the blower 196, for example according to a set of instructions stored on a memory device incorporated into the controller 120. In such embodiments, the blower 196 may be activated at a time and for a duration effective to draw fumes generated by the laser 115 without drawing power constantly or continuously.

Optionally, the system 200 may include a component configured to scan a machine-readable feature 144 included on the DBS card 140a. For example, in some embodiments, the camera 116 may be configured to obtain an image of the machine-readable feature 144, which then may be translated (e.g., by the controller 120) into information about the DBS card 140a and/or the subject who provided the blood sample stored on the DBS card 140a, such as the subject's name, the date the sample was obtained, a subject identification number (e.g., for blind trials or other subject-identification protective purposes), and/or the assay(s) to be performed.

Alternatively or in addition to the configurations described above, the controller 120 may be configured to store information about the DBS card 140a (e.g., identifying information about the subject and/or the blood sample), the location of the portion of the DBS 142 to be excised, the pattern of light beam to be executed by the laser 115, the power voltage and/or amperage supplied to the laser 115, the image obtained by the camera 116, notes from an operator of the system 200, and/or any error messages (e.g., power faults, laser faults, faults in transporting (e.g., feeding) the DBS card 140a through the components of the system 200, and/or possible defects in the DBS card 140a determined from the image obtained by the camera 116) that are generated by the system 200 during processing of the DBS card 140a.

### III. SELECTED CONFIGURATIONS OF DBS CARDS

The present technology also includes DBS cards including machine-readable identification feature which enables automated processing of multiple DBS cards and interpretation of the resulting assay data. In some embodiments, the DBS cards comprise a machine-readable identification feature such as a linear barcode, a matrix barcode (e.g., a QR code), an alphanumeric code, or other suitable type of machine-readable code.

Referring now to FIG. 3, a DBS card 340 suitable for use with systems and methods of the present technology includes DBSs 142a-e for depositing a blood sample from a subject. The DBS card 340 also includes standard features of DBS cards, such as a fold or fold line 341, a flap 343 under which the top edge of the DBS card 340 is secured for storage (optionally marked with a label 345), and space 346 for information about the subject, such as name, date of sampling, etc. The DBS cards 340 suitable for use with the systems and methods of the present technology may also include a machine-readable identification feature 344 which is readable by a component of the system 100/200 (e.g., the camera 116). The machine-readable feature 344 may be located at any suitable location on the DBS card 340 in which the code-reading component of the system 100/200 (e.g., the camera 116) can scan the machine-readable feature 344. In some embodiments, the machine-readable feature 344 is located on the same panel as the space 346 for information about the subject. In other embodiments, the machine-readable feature 344 is located on the same panel as the DBSs 142a-e (e.g., between the fold 341 and the flap 343). In another embodiment, the machine-readable feature 344 is located on the panel which is folded over the DBSs 142a-e during storage.

In embodiments in which the DBS card 340 includes the machine-readable feature 344, the system 100/200 may be configured to obtain an image (e.g., scan) of the machine-readable feature 344 and determine one or more operating parameters, such as the location of the cut to be made, the size of cut and/or light beam pattern to be executed by the laser 115, the type and/or location of the receptacle 150 to be used, and the like. For example, in some embodiments, the camera 116 may be configured to obtain an image of the machine-readable feature 344, which then may be translated (e.g., by the controller 120) into information about the DBS card 140a and/or the subject who provided the blood sample stored on the DBS card 140a, such as the subject's name, the date the sample was obtained, a subject identification number (e.g., for blind trials or other subject-identification protective purposes), and/or the assay(s) to be performed.

In some embodiments, the DBS card 140 is encased or at least partially enclosed in a protective container, such as a plastic case. In such embodiments, the system 100/200 may be configured to remove the DBS card 140 from the container before the system 100/200 excises a portion of a DBS 142 from the DBS card 140. In some embodiments, the system 100/200 is additionally configured to return the DBS 140 to its initial encased or at least partially enclosed configuration after the portion of the DBS 142 is excised. For example, system 200 may be configured to store a plurality of encased or at least partially enclosed DBS cards 140 in the DBS card hopper 260 in the encased or at least partially enclosed configuration, and the DBS card unfolder 270 is configured to expose at least a portion of the DBS cards 140 before the DBS card feeder 280 positions the exposed portion of the DBS card 140a for processing by the laser 115. The DBS card depository 290 may be configured to receive and return the processed DBS cards 140b to their initial encased or at least partially enclosed configuration before storage.

### IV. SELECTED METHODS FOR TOUCHLESS PROCESSING OF DBS CARDS

The present technology also includes methods for processing one or more DBS cards by cutting at least a portion of a DBS from each DBS card and depositing each cutting into a receptacle, wherein the method does not include physically contacting the DBS in order to minimize or eliminate a risk of cross-contaminating the dried blood samples (e.g., “touchless” processing).

In some embodiments, the method comprises positioning a DBS card in alignment with a receptacle, wherein the DBS card has at least one DBS comprising dried blood from a subject; contacting the DBS with a beam of light from a laser in a pattern sufficient to excise at least a portion of the DBS from the DBS card; and depositing the excised portion of the DBS into the receptacle.

In some embodiments, the method further comprises obtaining an image of the DBS card before contacting the DBS with the beam of light from the laser. In such embodiments, the image may include information about one or more of the DBSs on the DBS card, and/or a machine-readable code. In some embodiments, the pattern for the beam of light is determined by the system based at least in part on the image. Alternatively or in addition, the location of the DBS to be contacted with the beam of light is determined by the system based at least in part on the image. In embodiments wherein the DBS card comprises a plurality of DBSs, one of the plurality of DBSs may be selected to be contacted with the beam of light based at least in part on the image. In some embodiments, the method further comprises storing information comprising the location and/or the light beam pattern in association with the machine-readable code in a database. For example, the controller 120 of system 100 or system 200 may include a database configured to store information about the location and/or the light beam pattern in association with the machine-readable code corresponding to a DBS card.

The method may further comprise analyzing the excised portion of the DBS for the presence of one or more diseases. In some embodiments, the disease is HIV. In some embodiments, the disease is malaria. Any suitable method of analyzing the excised portion of the DBS may be used. For diseases detectable by analyzing blood for specific genetic material (e.g., viral or bacterial diseases), the analysis may include PCR, RT-PCR, LAMP, NASBA or other similar genetic amplification method known to those of skill in the art. In some embodiments, the result of the disease testing is stored in a database in association with the machine-readable code described above. For example, the controller 120 of system 100 or system 200 may include a database configured to store a test result in association with the machine-readable code corresponding to a DBS card.

In embodiments wherein the receptacle is housed in a receptacle support comprising a plurality of receptacles, the method may further comprise determining an assay to be performed on the DBS; selecting a receptacle from among the plurality of receptacles after positioning the DBS card; and (i) if the selected receptacle is in alignment with a first DBS having a sufficient area comprising dried blood for the determined assay, contacting the first DBS in alignment with the selected receptacle with a beam of light from the laser in a pattern sufficient to excise at least a portion of the first DBS from the DBS card, or (ii) if the selected receptacle is not in alignment with a DBS having a sufficient area comprising dried blood for the determined assay: (a) repositioning the DBS card and/or the receptacle to align a second DBS having a sufficient area comprising dried blood for the determined assay, and (b) contacting the second DBS in alignment with the selected receptacle with a beam of light from the laser in a pattern sufficient to excise at least a portion of the second DBS from the DBS card.

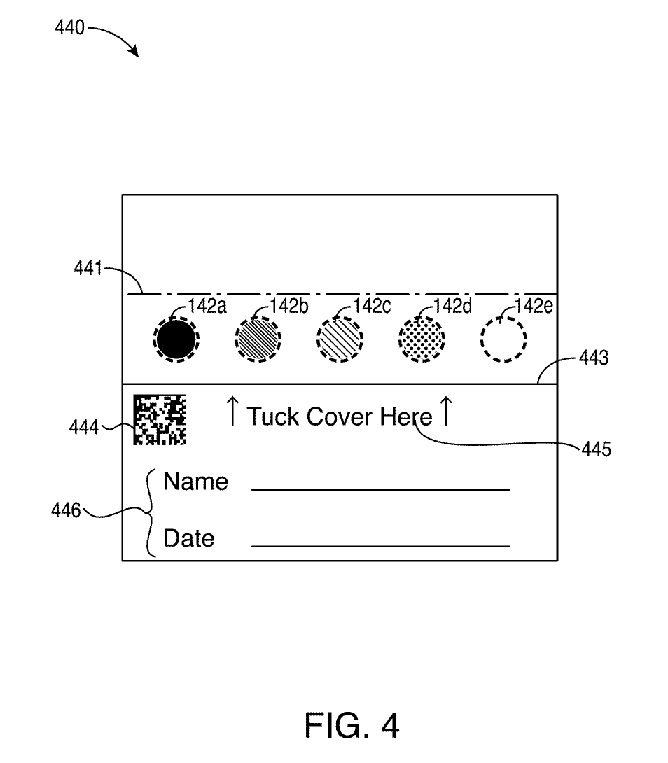

Methods of the present technology may further comprise processing a calibration DBS card. In such embodiments, the method may comprise providing a calibration DBS card including a plurality of calibration DBSs each having a different concentration of one or more analyte; positioning the calibration DBS card such that each calibration DBS is in alignment with a single receptacle; contacting each of the calibration DBSs with a beam of light from a laser in a pattern sufficient to excise at least a portion of each calibration DBS from the calibration DBS card; and depositing each of the excised portions of the calibration DBSs into the aligned receptacles. One example embodiment of a calibration DBS card 440 is shown in FIG. 4. In some embodiments, one of the calibration DBSs 142a-e may include no analyte (e.g., a negative control). The calibration DBS card 440 may include a machine-readable code 444 which may be stored in a database (e.g., included in the controller 120 of system 100 or system 200) in association with information about the locations and/or the patterns of light beam patterns executed by the laser 115 to excise a portion of each of the calibration DBSs 142a-e.

The present technology also provides methods for processing a plurality of DBS cards without touching any of the DBSs of the DBS cards (e.g., “touchless” automatic bulk processing of DBS cards). In some embodiments, the method comprises: (i) providing a plurality of DBS cards; (ii) selecting a first DBS card from the plurality of DBS cards, the first DBS card having at least one DBS comprising dried blood; (iii) positioning the first DBS card in alignment with a first receptacle; (iv) contacting the first DBS with a beam of light from a laser in a pattern sufficient to excise at least a portion of the first DBS from the first DBS card; (v) depositing the excised portion of the first DBS into the first receptacle; (vi) depositing the first DBS card into a DBS card depository; and (vii) depositing an excised portion of a second DBS into a second receptacle by repeating steps (ii) to (vi) for a second DBS card selected from the plurality of DBS cards.

In some embodiments, the method is suitable for efficiently analyzing a large number of subject samples simultaneously (e.g., “pooled” analysis), for example for high throughput screening of low-prevalence diseases. Typically, pooled screening methods include combining a large number of samples into a single pooled sample and then analyzing the combined samples for the presence of the target analyte of interest. If no analyte associated with the disease-causing organism is detected (e.g., no genetic material of a selected pathogen or virus is detected by PCR or RT-PCR), then none of the individual samples is likely to be infected with that pathogen. Accordingly, the method of the present technology may include pooling excised portions of multiple DBSs before analyzing the pooled spots for the presence of a disease. In such embodiments, the second receptacle configured to receive the excised portion of the second (and subsequent) DBS is the same as the first receptacle configured to receive the excised portion of the first DBS. The system 100/200 may be configured to pool a predetermined number of DBSs before providing a new receptacle to receive additional (optionally pooled) DBSs. For example, the controller 120 may be configured to pool 2 to about 5,000 samples (e.g., excised portions of DBSs), about 50 to about 2,500 samples, about 100 to about 2,000 samples, about 250 to about 1,000 samples, or about 500 to about 750 samples in a single receptacle. In such embodiments, the controller 120 may additionally be configured to store information about each DBS card, the location and/or the light beam pattern associated with each DBS card, for example by associating the stored information with a machine-readable code located on each processed DBS card. In some embodiments, the information is stored in a database incorporated in the controller 120.

In other embodiments, each DBS is processed into a separate receptacle. Such methods are useful, for example, in detecting the presence of a disease-associated analyte for diseases with relatively high incidence rates, or for re-analyzing individual DBS cards previously analyzed in a pooled method (e.g., as described above). In such embodiments, the second receptacle (and each subsequent receptacle) is separate from the first receptacle.

In any automated touchless processing method described herein, an image of the each DBS card may be obtained before contacting the DBS with the beam of light from the laser. In such embodiments, the image may include information about one or more of the DBSs on the DBS card, and/or a machine-readable code. In some embodiments, the image is used at least in part to determine a location of the first DBS to be contacted with the beam of light. In embodiments wherein the DBS card comprises a plurality of DBSs, the DBS is selected from the plurality of DBSs, based at least in part on the image, to be contacted with the beam of light. In some embodiments, the method further comprises storing information comprising the location and/or the light beam pattern for each or at least some of the DBS cards in association with the machine-readable codes in a database. For example, the controller 120 of the system 200 may include a database configured to store information about the location and/or the light beam pattern in association with the machine-readable code corresponding to a DBS card.

Alternatively, the system 200 may be configured to periodically obtain an image of only some of the plurality of DBS cards to be analyzed, such as for quality control assessments. In such embodiments, an image of the DBS card may be obtained before, during, and/or after the laser executes the light beam pattern. The image(s) may be stored for later review, such as in a database incorporated in the controller 120. In some embodiments, the system 200 is configured to provide a quality report based on the image(s). The quality report may be generated by the controller 120, and may be provided to a user in any suitable form, such as in a printout, in an electronic format, and/or displayed on a screen.

### V. EXAMPLES

### Example 1

### Detection of Malaria Infection

Malaria infection can be diagnosed by demonstrating the causative Plasmodium parasite in red blood cells by microscopy, by rapid antigen detection or by molecular methods. Microscopy is time consuming and not amenable to high-throughput use, and rapid antigen detection kits are insufficiently sensitive for many settings. While more sensitive, most molecular methods require larger sample volumes than DBS can accommodate to achieve sufficiently high sensitivity. A first-generation highly sensitive quantitative RT-PCR assay targeting the P. falciparum 18S rRNA from total nucleic acids demonstrated sensitive detection from only 50 μL of liquid whole blood (Murphy, S. C., et al., Real-time quantitative reverse transcription PCR for monitoring of blood-stage Plasmodium falciparum infections in malaria human challenge trials. Am. J. Trop. Med. Hyg., vol. 86(3), pages 383-94 (2012)). Since each parasite contains ˜3.4-4.0 log₁₀ RNA copies, the assay can detect as few as 20 parasites per mL of whole blood, similar to other high volume DNA-only assays used for vaccine trial monitoring. The small sample volume of the present assay afforded the possibility of using DBS for detecting patent and pre-patent (sub-microscopic) parasitemia.

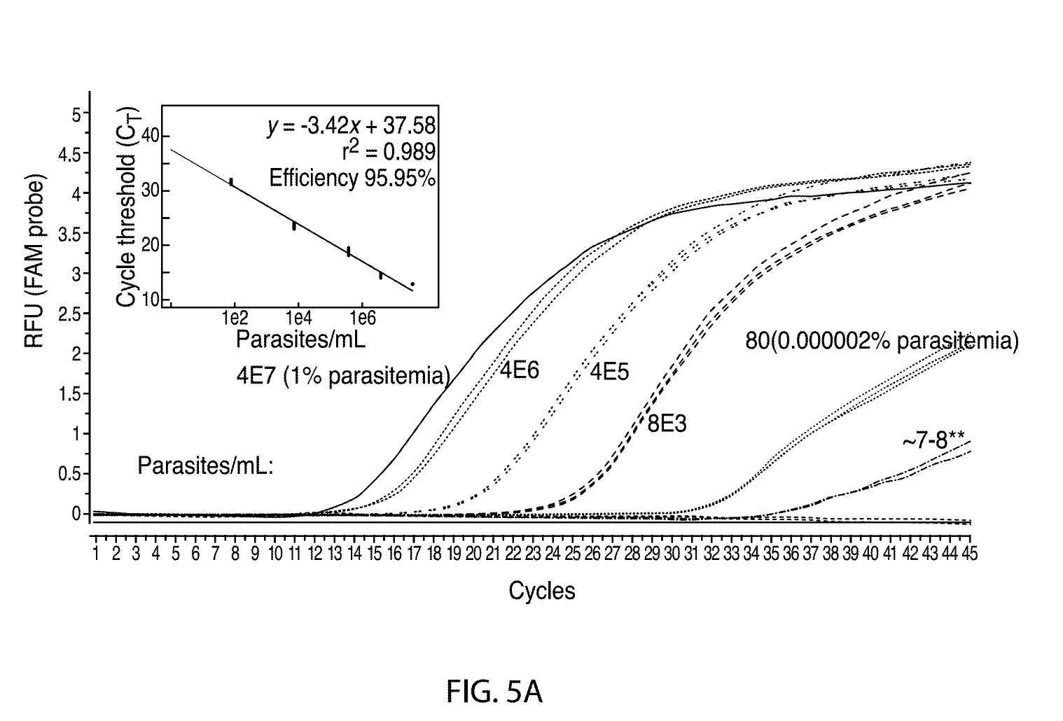

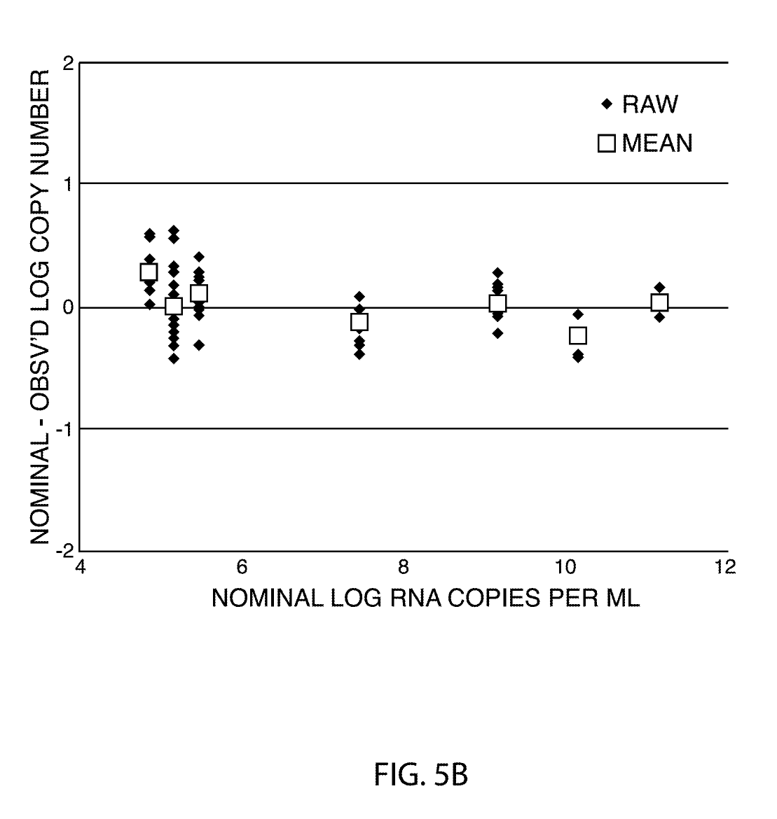

The first-generation RT-PCR assay described above was modified to use TaqMan probe chemistry on a high-throughput instrument as a second generation assay. Data generated from testing synthetic RNA standards (5×10² to 1×10⁹ copies per RT-PCR reaction) in an extracted whole blood internal control RNA-containing matrix were used to evaluate the standard curve, reportable range and carryover. Data generated from testing parasite-containing whole blood samples (4×10¹ to 4×10⁷ parasites per mL of blood) containing internal control RNA were used to evaluate accuracy, precision, analytical sensitivity, analytical specificity, reportable range and carryover. A ‘synthetic standard curve’ diluted in negative whole blood (data not shown) was compared against cultured blood-stage parasites in whole blood (FIG. 5A) to assess linearity of the liquid sample-based assay across a wide range of analyte concentrations (1.5×10³ to 1.5×10⁷ copies per reaction for RNA standards and 0.000002%-1% parasitemia for parasite standards) and to generate an m2000-specific conversion factor (3.56 log₁₀ 18S rRNA copies per parasite; median 3.56 log₁₀; 95% CI 3.51-3.61 log₁₀; n=74 samples) for use in calculating the number of parasites per mL of whole blood. The conversion factor value is slightly lower than for the first-generation assay (3.98 log₁₀ RNA copies per parasite), which may reflect differences in the culture conditions, extraction platforms and/or extraction of 25 μL rather than 50 μL of total whole blood. Archival samples validated on the original assay were also tested in this assay and all calculated results agreed between first- and second-generation assays (data not shown). To test target recovery, eluates from high parasite density samples (4×10⁵ parasites/mL) were retained, pooled, added to lysis buffer, re-extracted and tested by RT-PCR to determine the recovery. By this measure, mean recovery was 107% (95% CI 60-154%), indicating nearly complete target recovery (data not shown).

Parasite-containing specimens (high, medium and low concentration) and negative control specimens were tested in triplicate over a 5-day timespan to calculate diagnostic sensitivity and diagnostic specificity. There were no false positive or false negative results in this dataset. The difference between the nominal (expected) and observed estimates was determined and plotted against the nominal value. Of 54 samples in this data set, the average log₁₀ difference (bias) across all 54 samples was 0.143 log₁₀ RNA copies/mL (95% CI −0.379 to 0.367 log₁₀ RNA copies/mL) and no samples showed a difference in recovery >0.5 log₁₀ RNA copies/mL. The correlation across all samples was linear (r²=0.9911; slope 1.03) with no concentration-dependent differences in recovery based on a Bland-Altman plot (not shown, r²=0.1278). Within-run (repeatability) and between-run (within lab) precision was determined by testing triplicate high, medium, low and negative samples daily for 5 days judged against the in vitro standard curve from the overall validation. Intra- and inter-assay components of variation were calculated as described in S. C. Murphy, Am. J. Trop. Med. Hyg., vol. 86(3), pages 383-94 (2012). The standard deviation (log₁₀ copy number) and the percent coefficient of variation (% CV=Standard deviation/mean) are reported in Table 1. Based on repeated testing of samples containing 500 copies of the synthetic malaria control RNA per reaction (data not shown), the analytical sensitivity was determined to be 20 parasites/mL.

TABLE 1
Precision studies

| | | | | | Intra- | Inter- |
|---|---|---|---|---|---|---|
| | | | | | assay | assay |
| | | | Expected | Expected | % CV | % CV |
| | Samples | | parasites/ | RNA log₁₀ | (within | (within |
| Control | per run | # runs | mL | copies/mL | run) | lab) |
| +++ | 3 | 5 | 4 × 10⁷ | 9.6 | 0.81% | 2.25% |
| ++ | 3 | 5 | 8 × 10³ | 7.9 | 1.78% | 3.24% |
| + | 3 | 5 | 8 × 10¹ | 5.9 | 3.52% | 6.30% |

The reportable range (FIG. 5B) was determined by assaying high and low parasitemia specimens. 0.000001% to >1% parasitemia). Based on 84 samples, the average difference was +0.149 log₁₀ copies/mL, with four samples at the lowest template concentrations showing differences >0.5 log₁₀ copies per mL from the expected value (maximum difference 0.629 log₁₀ units). High positive samples were followed by negative samples to test for carryover. There was no cross-contamination between liquid blood samples in this validation.

### Example 2

### Laser Processing of DBS Eliminates Cross-Contamination of DBS Samples Tested for the Presence of Malaria

The assay described in Example 1 was adapted to utilize blood samples stored on DBS cards. However, because the dynamic range of the P. falciparum 18S rRNA assay [˜2×10⁵ copies/mL to 4×10¹² copies/mL (2×10¹ parasites/mL to 4×10⁸ parasites/mL) includes samples with much higher template concentrations than observed for HIV-1 assays, DBS samples for malaria testing could have been more prone to cross-contamination from hole punching than HIV-1 DBS. Indeed, analysis of DBS samples processed by hole punching showed a significant rate of false positives due to cross-contamination for malaria samples. To overcome this problem, a laser cutting approach described substantially as above was used to process DBS without touching the blood-containing sections of the DBS card. Cross-contamination was not observed for samples processed using the laser cutting approach as described below.

When processing DBS samples using conventional punching, numerous false positives were detected in samples originating from malaria-negative whole blood (Table 2). Such samples were processed after a high or medium concentration malaria-positive samples, indicating template cross-contamination. Despite using the conventional HIV-compatible approach of punching the entire circle into a sample tube using a standard office supply-type hole puncher then cleaning the puncher by punching five clean DBS sheets before proceeding to the next sample, carryover occurred in >50% of conventionally processed, sequentially tested malaria-positive (+, ++, +++) and malaria-negative (−) DBS samples 1 to 18 in the order shown in the left-most column (Table 2). All samples were first processed using the conventional punch method. All samples were then processed using a laser configured as described herein.

TABLE 2

| | | | | | Laser cut method | | |
|---|---|---|---|---|---|---|---|
| | | Conventional punch method | | | | Log₁₀ | |
| Sample | Malaria | | Log₁₀ copies | Calculated | | copies 8.9 mm | Calculated |
| No. | Presence | C_{T} | 12.0 mm spot | Parasites/mL | C_{T} | spot | Parasites/mL |
| 1 | +++ | 22.49 | 6.76 | 65,173 | 21.85 | 6.95 | 184,330 |
| 2* | − | 32.34 | 3.83 | 77 | ND | ND | ND |
| 3 | +++ | 21.79 | 6.96 | 105,249 | 21.75 | 6.98 | 197,394 |
| 4* | − | 37.81 | 2.20 | 2 | ND | ND | ND |
| 5 | +++ | 21.39 | 7.08 | 138,409 | 21.47 | 7.06 | 239,108 |
| 6* | − | 37.09 | 2.41 | 3 | ND | ND | ND |
| 7 | +++ | 19.98 | 6.96 | 103,338 | 20.10 | 6.92 | 173,978 |
| 8* | − | 41.99 | 0.48 | <1 | ND | ND | ND |
| 9 | +++ | 19.30 | 7.15 | 163,121 | 20.79 | 6.72 | 109,478 |
| 10* | − | 34.55 | 2.71 | 6 | ND | ND | ND |
| 11 | +++ | 19.59 | 7.07 | 134,265 | 20.82 | 6.71 | 107,296 |
| 12 | − | ND | ND | ND | ND | ND | ND |
| 13 | +++ | 19.33 | 7.15 | 159,869 | 20.73 | 6.74 | 113,978 |
| 14* | − | 32.73 | 3.24 | 20 | ND | ND | ND |
| 15 | +++ | 19.57 | 7.08 | 136,080 | 20.03 | 6.94 | 182,349 |
| 16* | − | 36.02 | 2.28 | 2 | ND | ND | ND |
| 17 | ++ | 27.18 | 5.36 | 2,627 | 27.5 | 5.27 | 3,850 |
| 18* | − | 35.45 | 2.90 | 9 | ND | ND | ND |
| 19 | ++ | 26.84 | 5.46 | 3,315 | 27.69 | 5.21 | 3,381 |
| 20* | − | 43.66 | ND | <1 | ND | ND | ND |
| 21 | ++ | 27.00 | 5.41 | 2,971 | 26.95 | 5.43 | 5,611 |
| 22 | − | ND | ND | 0 | ND | ND | ND |
| 23 | ++ | 25.36 | 5.39 | 2,791 | 25.89 | 5.23 | 3,568 |
| 24 | − | ND | ND | 0 | ND | ND | ND |
| 25 | ++ | 24.64 | 5.60 | 4,525 | 26.53 | 5.05 | 2,322 |
| 26 | − | ND | ND | 0 | ND | ND | ND |
| 27 | ++ | 24.77 | 5.56 | 4,147 | 26.82 | 4.96 | 1,911 |
| 28 | − | ND | ND | 0 | ND | ND | ND |

*Malaria-negative samples in which malaria was detected by the conventional punch method (e.g., negative samples that had been cross-contaminated).

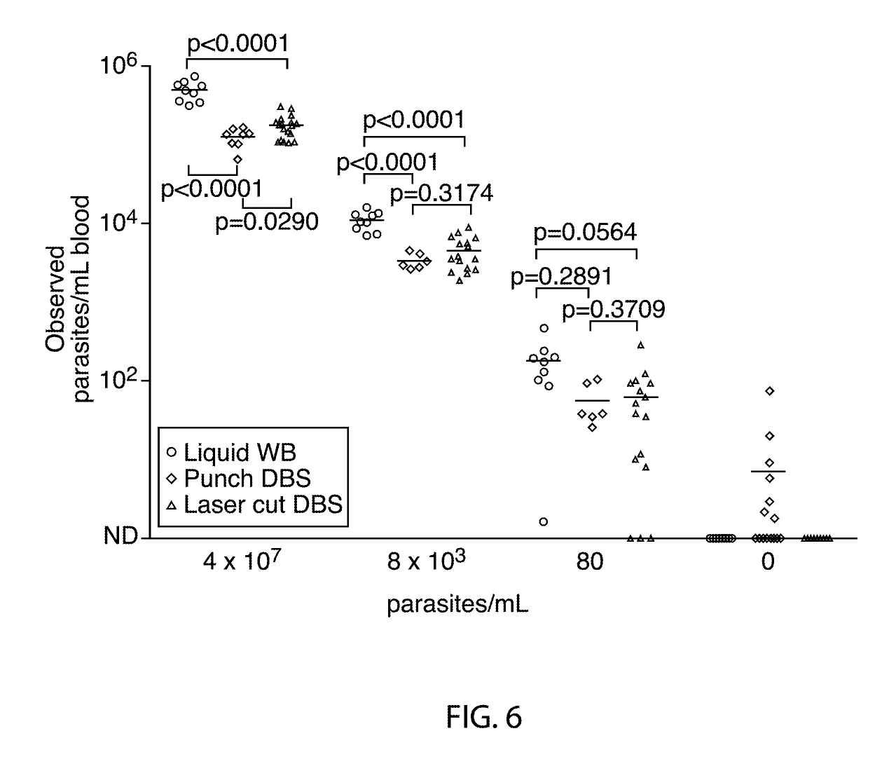

Compiled data on standardized whole blood samples processed by conventional liquid processing, by punch DBS processing or by laser cut DBS processing showed that only the punch processed DBS were susceptible to false positives. Of the samples that were processed by interspersing negative samples (0 parasites/mL) with high (4×10⁷ parasites/mL), medium (8×10³ parasites/mL) and low positive (80 parasites/mL) samples, cross-contamination was not detected in laser-cut DBS or conventional liquid samples, but was detected following punched samples amongst known negative samples in 7 of 8 instances following a high positive sample and in 2 of 6 instances following medium positive samples (Table 2; FIG. 6). Of the false positive, punch-processed DBS samples, 2/9 generated results of ≧20 parasites/mL (the limit of quantification for this assay) and all were due to contamination of at least 100 copies of contaminating template per sample (3500 copies/parasite), a concerning level of template easily detected by most molecular assays. Previous experience with liquid whole blood testing over several years did not reveal similar contamination issues. False positives due to cross-contamination would be problematic since the tests are routinely used in the days following malaria treatment, and occasionally detect the parasite template in the low positive range (<20 parasites/mL). When such low positives are detected, the patient must be followed with repeated testing to ensure that low positive results eventually drop to undetectable levels. Low positive results are therefore useful for monitoring the rise and fall of malaria parasite infection in exposed persons such that the presence of false positives due to cross-contamination would make such evaluations impossible.

Additional samples that are not included in Table 2 are also displayed in FIG. 6 and were tested in order to ascertain the recovery (quantitative agreement) between processing methods. Recovery did not differ between punched or laser-cut DBS, but was moderately reduced (˜0.5 log₁₀ copies/mL) for all DBS compared to liquid whole blood samples (FIG. 6). The mean differences between the liquid blood and punched or laser-cut DBS were −0.60 and −0.45 log₁₀ parasites/mL for high positives and −0.51 and −0.38 log₁₀ parasites/mL for medium positives, respectively. There were no significant differences between low positive liquid blood or DBS; p values were calculated using unpaired t tests. Similar losses were reported for HIV-1 DBS relative to liquid samples and may reflect degradation of the template on DBS or an inability to elute template from the DBS.

Since recovery for DBS samples was less than for liquid samples, DBS samples require a different calibration standard curve than liquid samples. A DBS-derived standard curve may therefore be obtained (for example using a standard DBS card as shown in FIG. 4) rather than liquid calibration standards since the DBS curve fully mimics that losses observed for clinical DBS samples.

Some laser-cut low positive samples were not detected by RT-PCR. In such instances, two laser-cut discs were processed in a single tube (equivalent to 54.9 microliters of whole blood) in an attempt to overcome this qualitative detection problem. Amongst low positive samples (80 parasites/mL) where this approach was used, 13 of 13 samples were positive (mean parasite density 117 parasites/mL; data not shown), which essentially overcame the false negatives observed when one disc was used. Since detection of low-positives was restored by using two laser cut discs per sample, the false negative findings depicted in FIG. 6 for low positive laser cut samples were most likely due to a limiting Poisson distribution of parasites (e.g., the actual presence or absence of an actual parasite on the single disc) and not due to an effect from laser cutting.

### Example 3

### Correlation Between Processed DBS Samples and Processed Liquid Blood Samples

A series of 108 de-identified samples collected from subjects in a clinical trial were tested using both the laser-cut DBS method of the present disclosure and a standard liquid blood (LB) method. The number of positive samples for each assay are shown below in Table 3.

TABLE 3

| Number of | | |
|---|---|---|
| positive samples | DBS+ | DBS− |
| LB+ | 33 | 9 |
| LB− | 4 | 62 |

The source samples were collected from subjects who were in the initial stages of malaria infection (e.g., the number of parasites was exceedingly low and near the limit of detection for the assay). The results show general agreement between laser-cut DBS cards and LB with 33/108 positive by both methods and 62/108 negative by both methods. The incongruent values likely have more to do with the very low parasite load (e.g., Poisson statistics affecting sampling proportions) than with actual false-positives and/or false-negatives resulting from defects in the liquid blood assay or the DBS processing assay.

### Example 4

### Processed DBS Samples for Detection of HIV-1

The performance of DBS whole blood collection and testing methods for detecting HIV-1 antibodies and HIV-1 nucleic acid (NA) in a population-based HIV surveillance study were assessed. Plasma and DBS were collected from multiple subject cohorts that included known HIV-negative and known HIV-positive subjects. Plasma is processed by standard methods. DBS samples are processed using a laser configured consistently with the present disclosure. Samples are analyzed by three HIV-1/2 molecular diagnostic tests and a fourth syphilis antibody test. A total of 1200 samples from phases I and II are analyzed.

A preliminary analysis was conducted to determine HIV-1 infection status on 316 finger-prick DBS cards collected in Chicago, Ill. between July 2013 and April 2014. DBS samples (50 μL) venous blood per spot) were obtained from HIV-1 viremic and HIV-1 seronegative patients. Samples were excised with the laser cutter as described herein. From each DBS card, one 50 μL spot was eluted in 0.5-mL phosphate buffered saline for HIV serological tests and another 50 μL spot was eluted in 2.5 mL BioMérieux NucliSENS lysis buffer for HIV NA testing. HIV-1 tests included the Abbott Architect HIV Ag/Ab Combo Assay: HIV-1/-2 Ab and HIV-1 Ag; the Bio-Rad Multispot HIV-1/HIV-2 Rapid Test: differentiation of HIV-1 and -2 Ab and the Abbott RealTime HIV-1 assay: quantification of HIV-1 NA. Analysis revealed that 185/316 subjects were HIV-negative, while 2/316 were acutely infected with HIV. 68/316 had low viremia established infection and 61/316 had high viremia established infection (Table 4). A subset of samples (33) were tested to compare whole blood DBS performance against plasma samples using the Abbott Architect HIV Ag/Ab Combo Assay, and all samples showed good quantitative concordance including an absence of false positives in the HIV-1 negative control subjects. The Abbott RealTime HIV-1 assay was determined to have a DBS sensitivity of 2000 copies HIV-1/mL whole blood using a single DBS sample. To improve the sensitivity of this assay, a two-spot assay was evaluated. Optimal two-spot assay performance was obtained by extracting the samples on the bioMerieux miniMag extraction instrument for subsequent quantification by the enzymatic amplification of Abbott RealTime HIV-1 assay—the sensitivity of this approach was 520 copies HIV-1/mL whole blood, which is sufficiently sensitive to detect WHO-defined virological failures at the defined threshold of 1000 HIV-1 RNA copies/mL plasma. Thus, laser-cut DBS provide an inexpensive and patient-friendly way to collect, store and transport patients' blood samples. Laser cut DBS can be tested for HIV-1/2 using the Abbott Architect HIV Ag/Ab Assay, Bio-Rad Multispot Rapid Test and Abbott RealTime HIV-1 assay.

TABLE 4

| HIV-1 | | | | |
|---|---|---|---|---|
| Infection | No of | Abbott HIV Ag/Ab | MultiSpot HIV- | Abbott RealTime HIV- |
| Status | cases | Combo | 1/HIV-2 Test | 1 assay |
| No infection | 185 | Non-reactive | Non-reactive | Not Detected |
| Acute | 2 | Reactive | Non-reactive | Detected (88900 and |
| infection | | (S/CO* = 4.07, and | | 4330000 c/mL blood) |
| | | 4.31) | | |
| Established | 68 | Reactive | Reactive (HIV-1) | Not detected (n = 62) or |
| infection, low | | (S/CO: median, 341; | | <2000 c/mL blood (n = 6) |
| viremia | | range, 14-4325) | | |
| Established | 61 | Reactive | Reactive (HIV-1) | ≧2000 c/mL blood |
| infection, | | (S/CO: median, 579; | | (median, 14300; range, |
| high viremia | | range 11-1000 | | 2150-330000) |

### Example 5

### Processed DBS Samples for Detection of HIV-1

As part of a large collaborative project, samples were collected from up to 4100 HIV-positive patients receiving antiretroviral therapies (ART) in 15 health facilities across Uganda. HIV-1/-2 viral loads were analyzed and the data used as part of a broad ART cost-effectiveness study in Uganda. Blood samples were transported from the rural health facilities to the nearest urban health facilities for refrigeration and then transported onward to a central laboratory in Kampala for plasma viral load testing. Dried blood spots (DBS) were transported to a central laboratory and then transferred to our facility (University of Washington) for DBS viral load testing. DBS were cut using the laser cutter as described in Murphy et al. 2012, and a two-spot sample was extracted using the bioMérieux miniMag system and quantified using the Abbott RealTime HIV-1 assay; for comparison, the viral load from plasma samples were measured entirely by the FDA-approved Abbott RealTime HIV-1 assay. To date, >1300 samples have been tested. When the study is completed, the laser cut DBS viral load results will be compared to the corresponding plasma viral load values.

As of June 2014, 1349 samples have been processed using the laser cutting system and subsequently tested for whole blood viral load and have a paired plasma sample that was separately tested by the FDA-approved assay. Interim analysis of these samples alone showed that 135/192 plasma-positive samples were also DBS-positive for HIV and that 1122/1157 plasma-negative samples were also DBS-negative for HIV (Table 5). Using the WHO-defined indicator of virological failure (≧1000 copies/mL plasma) as the sensitivity cutoff, the DBS approach had a sensitivity of 70.3% and a specificity of 97.0%.

TABLE 5

| | DBS+ | DBS− | Total |
|---|---|---|---|
| Plasma + | 135 | 57 | 192 |
| Plasma − | 35 | 1122 | 1157 |
| Total | 170 | 1179 | 1349 |

Performance characteristics of the DBS viral load assay are shown in Table 6.

TABLE 6

| | |
|---|---|
| VL cutoff | 1000 |
| Sensitivity | 0.703 |
| Specificity | 0.970 |
| PPV | 0.794 |
| NPV | 0.952 |

“PPV” = positive predictive value;
“NPV” = negative predictive value.

Examples 4 and 5 demonstrate that the laser cutting system disclosed herein provides rapid excision of samples. A slightly modified racking system currently in use allows the technologist to deposit two laser cut DBS discs into a single tube for onward use in the two-spot assay. This modification is facilitated by moving the destination tube independent of the static DBS card, although either component could be moved relative to the other in future generations of laser cutting DBS devices.

### VI. CONCLUSION

This disclosure is not intended to be exhaustive or to limit the present technology to the precise forms disclosed herein. Although specific embodiments are disclosed herein for illustrative purposes, various equivalent modifications are possible without deviating from the present technology, as those of ordinary skill in the relevant art will recognize. In some cases, well-known structures and functions have not been shown or described in detail to avoid unnecessarily obscuring the description of the embodiments of the present technology. Although steps of methods may be presented herein in a particular order, alternative embodiments may perform the steps in a different order. Similarly, certain aspects of the present technology disclosed in the context of particular embodiments can be combined or eliminated in other embodiments. While advantages associated with certain embodiments of the present technology may have been disclosed in the context of those embodiments, other embodiments can also exhibit such advantages, and not all embodiments need necessarily exhibit such advantages or other advantages disclosed herein to fall within the scope of the present technology. Accordingly, this disclosure and associated technology can encompass other embodiments not expressly shown or described herein.

Throughout this disclosure, the singular terms “a,” “an,” and “the” include plural referents unless the context clearly indicates otherwise. Similarly, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the terms “comprising” and the like are used throughout to mean including at least the recited feature(s) such that any greater number of the same feature and/or additional types of other features are not precluded. Directional terms, such as “upper,” “lower,” “front,” “back,” “vertical,” and “horizontal,” may be used herein to express and clarify the relationship between various elements. It should be understood that such terms do not denote absolute orientation. Reference herein to “one embodiment,” “an embodiment,” or similar formulations means that a particular feature, structure, operation, or characteristic described in connection with the embodiment can be included in at least one embodiment of the present technology. Thus, the appearances of such phrases or formulations herein are not necessarily all referring to the same embodiment. Furthermore, various particular features, structures, operations, or characteristics may be combined in any suitable manner in one or more embodiments.

1. A system for processing a dried blood spot (DBS) card, the system comprising:
a DBS card support configured to position a DBS card in a first orientation;
a laser positioned proximate to the DBS card;
a receptacle support configured to position a receptacle below the DBS; and
a controller operably connected to the laser and configured to cause the laser to cut at least a portion of the DBS from the DBS card.

2. The system of claim 1 further comprising a camera, wherein the system is configured to:
obtain an image of one or more DBSs on the DBS card using the camera;
determine a location and shape of a cutting pattern based at least in part on the image; and
cause the laser to cut a DBS corresponding to the location and shape of the determined cutting pattern.

3. The system of claim 1, wherein the receptacle support is configured to position a sample tube below at least one DBS.

4. The system of claim 1, wherein the receptacle support is configured to position a well of a multi-well plate below at least one DBS.

5. The system of claim 1, wherein the laser is configured to move relative to the DBS card.

6. The system of claim 1 further comprising a mirror configured to adjustably reflect a beam from the laser onto the DBS card.

7. The system of claim 1 further comprising an exhaust positioned over the DBS card.

8. The system of claim 7 further comprising a housing including the laser and the exhaust.

9. The system of claim 7, wherein the DBS card support includes the exhaust.

10. The system of claim 1, wherein the DBS card support includes at least one pair of DBS card mounts.

11. The system of claim 1, wherein the DBS card support is configured to support the DBS card at an adjustable or selectable distance above the receptacle.

12. The system of claim 11, wherein the DBS card support includes a plurality of DBS card mounts positioned at a plurality of distances above the receptacle.

13. The system of claim 1, wherein the DBS card support and the receptacle support are integrated into a single DBS card and receptacle support.

14. The system of claim 1, wherein the system is configured to cut and deposit the at least portion of the DBS in the receptacle without contacting the DBS.

15. The system of claim 1, wherein the DBS card support is configured to support the DBS card at an adjustable or selectable distance from the laser.

16. The system of claim 1, wherein the laser is configured to be positioned above the DBS card at an adjustable or selectable distance.

17. The system of claim 2, wherein the DBS card further comprises a machine-readable feature, and wherein the camera is configured to scan the machine-readable feature.

18. The system of claim 17, wherein the machine-readable feature comprises a bar code.

19. The system of claim 17, wherein the machine-readable feature comprises a QR code.

20. The system of claim 17, wherein in the controller is configured to store the machine-readable feature, the location of the determined cutting pattern, and the shape of the determined cutting pattern.

21. The system of claim 2, wherein the determined location and shape of the cutting pattern corresponds to a predetermined area of the DBS to be cut from the DBS card.

22. The system of claim 21, wherein the predetermined area is about 10 mm² to about 100 mm².

23. The system of claim 21, wherein the predetermined area is about 50 mm² to about 75 mm².

24. A system for automatically processing a plurality of DBS cards, the system comprising:
a laser configured to cut at least a portion of a DBS;
a DBS card hopper configured to store a plurality of DBS cards;
a DBS card support configured to position a DBS card in a first orientation proximate to the laser;
a DBS card feeder operably connected to the DBS card hopper and the DBS card support, the DBS card feeder configured to select a single DBS card from the DBS card feeder and feed the DBS card to the DBS card support;
a receptacle support configured to position a receptacle below the DBS;
a DBS card depository operably connected to the DBS card support and configured to receive the DBS card from the DBS card support; and
a controller operably connected to the laser, the DBS card feeder and the receptacle support, the controller configured to:
cause the DBS card feeder to select the DBS card from the DBS card hopper and feed the DBS card to the DBS card support,
cause a receptacle to be positioned under a DBS of the DBS card,
cause the laser to cut at least a portion of the DBS from the DBS card, and
cause the DBS card to be deposited in the DBS card depository.

25. The system of claim 24, wherein the laser and the receptacle support are each configured to move relative to the DBS card support.

26. The system of claim 24, wherein the laser and the DBS card support are each configured to move relative to the receptacle support.

27. The system of claim 24, wherein the DBS card support and the receptacle support are each configured to move relative to the laser.

28. The system of claim 24, further comprising a mirror configured to adjustably reflect a beam from the laser onto the DBS card.

29. The system of claim 24 further comprising a camera, wherein the system is configured to:
obtain an image of one or more DBSs on the DBS card using the camera;
determine a location and shape of a cutting pattern based at least in part on the image; and
cause the laser to cut a DBS corresponding to the location and shape of the determined cutting pattern.

30. The system of claim 24 further comprising an exhaust configured to receive a vapor produced by a beam emitted by the laser contacting the DBS card.

31. The system of claim 30, wherein the controller is operably connected to a blower configured to vent air through the exhaust.

32. The system of claim 24, wherein the DBS card support is configured to support the DBS card at an adjustable or selectable distance above the receptacle.

33. The system of claim 24, wherein the DBS card support is configured to support the DBS card at an adjustable or selectable distance from the laser.

34. The system of claim 24, wherein the laser is configured to be positioned above the DBS card at an adjustable or selectable distance.

35. The system of claim 29, wherein the controller is configured to:
determine the location and shape of the cutting pattern based at least in part on the image;
position the determined location in alignment with a beam from the laser; and
cause the laser to cut the DBS in the shape of the determined cutting pattern.

36. The system of claim 24, wherein the controller is configured to:
cause the DBS card feeder to select a second DBS card from the DBS card hopper and feed the second DBS card to the DBS card support,
cause a receptacle to be positioned under a DBS of the second DBS card,
cause the laser to cut at least a portion of the DBS from the second DBS card, and
cause the second DBS card to be deposited in the DBS card depository.

37. The system of claim 24, wherein the receptacle comprises a tube.

38. The system of claim 24, wherein the receptacle comprises a multi-well plate.

39. The system of claim 29, wherein the DBS card further comprises a machine-readable feature, and wherein the camera is configured to scan the machine-readable feature.

40. The system of claim 39, wherein the machine-readable feature comprises a bar code.

41. The system of claim 39, wherein the machine-readable feature comprises a QR code.

42. The system of claim 39, wherein in the controller is configured to store the machine-readable feature, the location of the determined cutting pattern, and the shape of the determined cutting pattern.

43. The system of claim 29, wherein the determined location and shape of the cutting pattern corresponds to a predetermined area of the DBS to be cut from the DBS card.

44. The system of claim 43, wherein the predetermined area is about 10 mm² to about 100 mm².

45. The system of claim 43, wherein the predetermined area is about 50 mm² to about 75 mm².

46. A method of processing a DBS card, the method comprising:
positioning a DBS card in alignment with a receptacle, the DBS card having at least one DBS comprising dried blood from a subject;
contacting the DBS with a beam of light from a laser in a pattern sufficient to excise at least a portion of the DBS from the DBS card; and
depositing the excised portion of the DBS into the receptacle.

47. The method of claim 46 further comprising obtaining an image of the DBS card before contacting the DBS with the beam of light from the laser.

48. The method of claim 47 further comprising determining the pattern based at least in part on the image.

49. The method of claim 47 further comprising determining a location of the DBS to be contacted with the beam of light based at least in part on the image.

50. The method of claim 47, wherein the DBS card comprises a plurality of DBSs, and wherein one of the plurality of DBSs is selected to be contacted with the beam of light based at least in part on the image.

51. The method of claim 50, wherein the DBS card comprises a plurality of DBSs, and wherein one of the plurality of DBSs is selected to be contacted with the beam of light based at least in part on the image.

52. The method of claim 47, wherein the image comprises information about one or more DBSs.

53. The method of claim 47, wherein the image comprises a machine-readable feature.

54. The method of claim 53 further comprising storing information comprising a location and/or the pattern associated with the machine-readable feature in a database.

55. The method of claim 46, wherein the excised portion of the DBS is analyzed for the presence of one or more diseases and/or pathogens.

56. The method of claim 55, wherein the one or more diseases and/or pathogens is associated with HIV and/or malaria.

57. The method of claim 55, wherein the analysis comprises PCR, RT-PCR, LAMP or NASBA.

58. The method of claim 46, wherein the receptacle is housed in a receptacle support comprising a plurality of receptacles, the method further comprising
determining an assay to be performed on the DBS;
selecting a receptacle from among the plurality of receptacles after positioning the DBS card; and
(i) if the selected receptacle is in alignment with a first DBS having a sufficient area comprising dried blood for the determined assay, contacting the first DBS in alignment with the selected receptacle with a beam of light from the laser in a pattern sufficient to excise at least a portion of the first DBS from the DBS card, or
(ii) if the selected receptacle is not in alignment with a DBS having a sufficient area comprising dried blood for the determined assay:
(a) repositioning the DBS card and/or the receptacle to align a second DBS having a sufficient area comprising dried blood for the determined assay, and
(b) contacting the second DBS in alignment with the selected receptacle with a beam of light from the laser in a pattern sufficient to excise at least a portion of the second DBS from the DBS card.

59. The method of claim 46 further comprising processing a calibration DBS card, the processing comprising:
providing a calibration DBS card comprising a plurality of calibration DBSs each having a different concentration of one or more analyte;
positioning the calibration DBS card such that each calibration DBS is in alignment with a single receptacle;
contacting each of the calibration DBSs with a beam of light from a laser in a pattern sufficient to excise at least a portion of each calibration DBS from the calibration DBS card; and
depositing each of the excised portions of the calibration DBSs into the aligned receptacles.

60. The method of claim 59, wherein one of the calibration DBSs includes no analyte.

61. The method of claim 59, wherein the calibration DBS card includes a machine-readable feature.

62. The method of claim 61 further comprising storing information comprising a location and/or the pattern associated with each of the calibration DBSs and with the machine-readable feature in a database.

63. A method of processing a plurality of DBS cards, the method comprising:
(i) providing a plurality of DBS cards;
(ii) selecting a first DBS card from the plurality of DBS cards, the first DBS card having at least one DBS comprising dried blood;
(iii) positioning the first DBS card in alignment with a first receptacle;
(iv) contacting the first DBS with a beam of light from a laser in a pattern sufficient to excise at least a portion of the first DBS from the first DBS card;
(v) depositing the excised portion of the first DBS into the first receptacle;
(vi) depositing the first DBS card into a DBS card depository; and
(vii) depositing an excised portion of a second DBS into a second receptacle by repeating steps (ii) to (vi) for a second DBS card selected from the plurality of DBS cards.

64. The method of claim 63, wherein the second receptacle is the same as the first receptacle.

65. The method of claim 63, wherein the second receptacle is separate from the first receptacle.

66. The method of claim 63 further comprising obtaining an image of the first and/or second DBS card before contacting the first or second DBS with the beam of light from the laser.

67. The method of claim 66 further comprising determining a location of the first DBS to be contacted with the beam of light based at least in part on the image.

68. The method of claim 66, wherein the first DBS card comprises a plurality of DBSs, and wherein the first DBS is selected from the plurality of DBSs, based at least in part on the image, to be contacted with the beam of light.

69. The method of claim 66, wherein the second DBS card comprises a plurality of DBSs, and wherein second DBS is selected from the plurality of DBSs, based at least in part on the image, to be contacted with the beam of light.

70. The method of claim 66, wherein the image comprises information about one or more DBSs on the DBS card.

71. The method of claim 66, wherein the image comprises a machine-readable feature.

72. The method of claim 71 further comprising storing information comprising a location and/or the pattern associated with the machine-readable feature in a database.

73. The method of claim 63, wherein the excised portion of the first DBS is analyzed for the presence of one or more diseases and/or pathogens.

74. The method of claim 73, wherein the one or more diseases and/or pathogens is associated with HIV and/or malaria.

75. The method of claim 73, wherein the analysis comprises PCR, RT-PCR, LAMP or NASBA.

76. The method of claim 63 further comprising processing a calibration DBS card, the processing comprising:
providing a calibration DBS card comprising a plurality of calibration DBSs each having a different concentration of one or more analyte;
positioning the calibration DBS card such that each calibration DBS is in alignment with a single receptacle;
contacting each of the calibration DBSs with a beam of light from a laser in a pattern sufficient to excise at least a portion of each calibration DBS from the calibration DBS card; and
depositing each of the excised portions of the calibration DBSs into the aligned receptacles.

77. The method of claim 76, wherein one of the calibration DBSs includes no analyte.

78. The method of claim 76, wherein the calibration DBS card includes a machine-readable feature.

79. The method of claim 78 further comprising storing information comprising a location and/or the pattern associated with each of the calibration DBSs and with the machine-readable feature in a database.
